(19) United States
(12) Patent Application Publication
(10) Pub. No.: US 2004/0054419 A1
(43) Pub. Date: Mar. 18, 2004

(54) System for trial implantation of a femoral hip prosthesis

(76) Inventors: Michael A. Serra, Cameron Park, CA (US); Shaun B. Hanson, Phoenixville, PA (US); Alfred S. Despres, Shingle Springs, CA (US)

Correspondence Address: Mark J. Pandiscio, Pandiscio & Pandiscio, P.C., 470 Totten Pond Road, Waltham, MA, 02451-1914, US

(21) Appl. No.: 10/434,572

(22) Filed: May 9, 2003

Related U.S. Application Data
(60) Provisional application No. 60/378,989, filed on May 9, 2002.

(51) Int. Cl. A61F 2/36

(52) U.S. Cl. 623/22.42; 623/22.46

(57) ABSTRACT

This invention provides for a system of temporary or “trial” implants which are used to establish the optimal location of the femoral head relative to the body of the hip stem implant. Using a series of interchangeable modular femoral neck components that mate with a series of interchangeable modular femoral body components, the location of the femoral head can be independently adjusted with respect to horizontal offset, vertical offset and anteversion angle relative to the body.

### REFERENCE TO PENDING PRIOR PATENT APPLICATION

This patent application claims benefit of pending prior U.S. Provisional Patent Application Serial No. 60/378,989, filed May 9, 2002 by Michael A. Serra et al. for MODULAR IMPLANT TRIALING SYSTEM AND METHOD (Attorney's Docket No. HAYES-9 PROV), which patent application is hereby incorporated herein by reference.

### FIELD OF THE INVENTION

This invention relates to medical apparatus and procedures in general, and more particularly to medical apparatus and procedures relating to total hip joints.

### BACKGROUND OF THE INVENTION

The aim of Total Hip Arthroplasty (“THA”) is the reduction of pain by restoring the form and function of a hip joint damaged by either trauma or disease. This is accomplished using engineered materials to construct an implantable device for the restoration of the joint mechanics and geometry, whereby the affected tissue is removed and replaced by the implantable device. Successful outcomes depend largely on the proper sizing, placement and orientation of the implant. Incorrect biomechanics (e.g., joint reaction forces, soft tissue balancing, leg length, etc.) can slow or prevent healing, cause gait abnormalities, result in dislocation of the joint, and lead directly to early implant failure, among other things.

The restoration of proper joint mechanics depends largely on good surgical technique, implants which are anatomically matched to the needs of the patient, and effective instrumentation for bony preparation, size and shape determination, and insertion of the final implant construct.

Prior to insertion of the actual implant, it is generally desirable to use a mock implant or “trial” as a means of evaluating the correct size and positioning of the implant within the bony canal. The surgeon implants the trial, reduces the trial ball and socket joint and evaluates joint stability, leg length, and range of motion (“ROM”), all of which depend, among other things, on the location of the femoral head relative to the body of the implant. The distance from the head of the implant to the body of the implant is generally referred to as “neck length”.

The proximal end of a normal femur (i.e., the “proximal femur”) has a gradual anterior twist to it so that the orientation of the endosteal envelope (i.e., inner bone geometry) gradually rotates externally. This moves the head anteriorly in the horizontal plane. In the horizontal plane, the included angle between the long axis of the neck and the body is commonly referred to as “anteversion”. When replacing a malformed hip joint, the surgeon may need to change the patient's natural anteversion to create proper and stable biomechanics of the hip joint. This process of “trialing” is often iterative as the surgeon tries different trial implants until the satisfactory joint mechanics are achieved by altering the neck length and anteversion.

The stability and range of motion (“ROM”) of the hip joint is achieved by placing the prosthetic femoral head at an orientation and distance from the proximal femur that allows for a normal range of motion without impingement of the hip onto the acetabulum. This impingement can be either implant-on-implant or implant-on-bone. The tension of the joint, which provides stability, is achieved by adjusting the neck length of the implant. Neck length can be adjusted vertically and horizontally. The anteversion angle can be adjusted rotationally about the vertical axis (i.e., about the long axis of the femur).

Increasing only the horizontal offset of the femoral head increases the tension of the tendons and muscles that attach the proximal femur to the pelvis without changing leg length. Increasing only the vertical offset of the femoral head increases the tension of the tendons and muscles that attach the proximal femur to the pelvis and changes leg length. Changing the anteversion of the femoral neck relative to the implant body directly affects the range of motion limits (without impingement or dislocation) with respect to the resting position of the femur.

Hip implant instrument systems generally provide some form of modular trial set. The most basic systems include a few trial femoral heads that allow the surgeon to vary the position of the head center along the axis of the neck (FIG. 1), altering the head offset in both the horizontal and vertical directions simultaneously. Slightly more advanced systems provide a combination of trial femoral heads with two or three trial neck components that allow pure horizontal variability. Better still are systems that offer independent horizontal and vertical control in addition to the trial heads (FIG. 2). The most advanced systems on the market, of which there are only a few, provide an additional degree of freedom by allowing modifications to the anteversion angle about the stem axis (FIG. 3). A trialing system of this type, with four independent degrees of freedom (i.e., translation along the neck axis, horizontal offset, vertical offset, and anteversion angle), gives the surgeon tremendous flexibility in positioning the head center, thus providing the best opportunity for optimization of the joint biomechanics.

The anteversion angle can be changed in either discrete, indexable jumps (e.g., 10 degree increments) or with infinitely fine, continuous movement. Infinite variability is highly desirable because (1) small rotational adjustments (e.g., less than about 15 degrees) are required in the vast majority of cases and (2) even rotational changes as small as about 5 degrees can affect impingement, dislocation rate, and the range of motion.

Using a system with four degrees of freedom, the surgeon must be able to quickly make adjustments in a quantifiable and repeatable way. At any point in the trialing process, a surgeon must have the option of independently adjusting neck length, horizontal offset, vertical offset, and/or the anteversion angle.

For example, a surgeon may be satisfied with the range of motion of the current trial, but may feel that the joint is vulnerable to dislocation because the soft tissue tension is too low. An increase in soft tissue tension can then be achieved by adjusting the horizontal or vertical offsets. However, if, as part of this adjustment of offset, the previously-set anteversion angle changes, the range of motion will be altered and the whole process must generally be repeated.

As another example, the horizontal and vertical offsets may be correct (i.e., the desired leg length and soft tissue balance may have been achieved) but the neck of the implant may impinge on the cup. This impingement will generally restrict the range of motion, increase wear debris, create an uncomfortable sensation for the patient, cause dislocations, lead to early loosening, and/or predispose the implant to early failure. The surgeon must be able to determine the current anteversion angle and make whatever adjustments are deemed necessary. Without a specific angle to reference, the surgeon must “eyeball” the change, thus making fine adjustments extremely difficult to achieve. In addition, without an accurate reference point for guidance, the trial and error process frequently becomes so cumbersome that the surgeon may be forced to settle on a “close enough” construct.

FIG. 4 shows a currently marketed, competitive modular trial system. This system is supplied with a series of neck components 1, each having a unique combination of horizontal and vertical offsets. The neck 1 has a cylinder 25 that mates with the straight bore 4 of the body 2 and is held in place with the locking screw 3. When loosely connected, the neck is able to spin about the stem axis, allowing adjustment to the desired anteversion angle. When forcibly connected, the construct is secure and the anteversion angle is locked. A disadvantage of this system is that the head center can only be adjusted by replacing the current neck component with one having the desired dimensions. The construct must therefore be loosened, causing the previously-established anteversion angle to be lost. After switching neck components, the surgeon is then forced to estimate the original anteversion angle before again tightening the screw.

FIGS. 5A and 5B show another currently marketed, competitive modular trial system. This system, too, is supplied with a variety of neck components. Each neck 5 has a serrated surface 7 that mates with the serrated surface 8 of the body 6. When engaged, the anteversion angle is locked by the interference of the serrated surfaces 7 and 8. As with the previous example (i.e., the system shown in FIG. 4), the system shown in FIGS. 5A and 5B suffers from the need to change neck components in order to vary offsets. If a surgeon wishes to adjust leg length or modify soft tissue tension, the surgeon must first release and remove the locking bolt 9, remove the current neck 5, insert a new neck 5, estimate the original anteversion position and then re-tighten the bolt. The system shown in FIGS. 5A and 5B has the further disadvantage of having a discrete or “indexable” anteversion angle adjustment due to the serration locking elements 7 and 8 (FIGS. 5A and 5B).

No implant instrument system currently available offers variability of the anteversion angle coupled with a system for locking the anteversion angle while allowing further vertical and horizontal adjustment.

No implant instrument system currently available offers independent anteversion angle variability coupled with independent control of vertical offset and horizontal offset.

### SUMMARY OF INVENTION

These and other objects are achieved by the present invention, which comprises a trial implant system that allows for independent control of horizontal offset, vertical offset and anteversion angle.

The novel trial implant system allows the anteversion angle to be locked, and then provides for further adjustment in either the horizontal and/or vertical offsets without disrupting the anteversion angle which has been locked.

In one form of the present invention, there is provided a modular femoral hip trial implant device for providing independent adjustment of the anteversion angle, vertical offset, and horizontal offset thereof, the modular trial implant comprising: a trial body component having a proximal end and a distal end, the trial body component defining a longitudinal axis between the proximal end and the distal end, the distal end of the trial body component being configured for placement within a bore in a bone, and a hub connection site configured at the proximal end of the trial body; a hub component having a proximal end and a distal end, the hub defining a longitudinal axis between the proximal end and the distal end, a neck component connection site configured at the proximal end of the hub component, a trial body component connection site configured at the distal end of the hub component, and an anteversion angle adjustment mechanism for selectively adjusting the hub component from a first orientation to a second orientation, wherein the neck component connection site is adjusted from a first radial location to a second radial location with respect to its rotational position about the longitudinal axis of the trial body; and a neck component having a proximal end and a distal end, a hub component connection site configured at the distal end of the neck component, and the proximal end of the neck component configured to receive a head thereon.

In another form of the present invention, there is provided a system for trial implantation of a modular femoral hip implant, the system providing independent adjustment of an anteversion angle, a horizontal offset and a vertical offset of the modular femoral implant, the system comprising: a plurality of trial body components, each one of the trial body components having a proximal end and a distal end, the trial body component defining a longitudinal axis between the proximal end and the distal end, the distal end of the trial body component being configured for placement within a bore in a bone, and a hub connection site configured at the proximal end of the trial body; a plurality of hub components, each one of the hub components having a proximal end and a distal end, the hub defining a longitudinal axis between the proximal end and the distal end, a neck component connection site configured at the proximal end of the hub component, a trial body component connection site configured at the distal end of the hub component, and an anteversion angle adjustment mechanism for selectively adjusting the hub component from a first orientation to a second orientation, wherein the neck component connection site is adjusted from a first radial location to a second radial location with respect to its rotational position about the longitudinal axis of the trial body; and a plurality of neck components, each one of the neck components having a proximal end and a distal end, a hub component connection site configured at the distal end of the neck component, and the proximal end of the neck component configured to receive a head thereon; wherein one from the plurality of trial body components is selected based on a geometry of a patient, one from the plurality of hub components is selected based on a geometry of a patient, and one from the plurality of neck components is chosen based on a geometry of a patient.

In another form of the present invention, there is provided a system for trial implantation of a femoral hip prosthesis which allows for independent establishment and adjustment of anteversion, horizontal offset, and vertical offset, the system comprising a plurality of trial bodies, a hub, a plurality of trial necks and a plurality of trial femoral heads, wherein each of the trial bodies register in a preformed cavity in the bone, and has a receiving mechanism for receiving the hub; wherein the hub is configured to be rigidly fixed to one of the trial bodies in a plurality of rotational positions with respect to the trial body, and comprises a receiving mechanism for receiving a trial neck, wherein each of the trial necks includes a mechanism for attaching to the hub, and has a geometry that reproduces the one of the available horizontal and vertical neck offsets available for implantation, and includes a mechanism for receiving the trial femoral heads, and wherein each of the plurality of trial femoral heads has a mechanism for attaching to the trial necks, and has a geometry that reproduces the neck offsets of the femoral head implants.

In another form of the present invention, there is provided a method for trial implantation of a femoral hip prosthesis, comprising: providing a trial implant system comprising: a series of trial bodies incrementally sized for a range of patients; a hub that can be fixed to the trial body in a variety of rotational positions; a series of trial neck components that attach to the hub, wherein the necks are available in a variety of dimensional combinations that allow for incremental adjustment of horizontal offset, vertical offset or a combination of both horizontal offset and vertical offset; and a series of trial femoral heads that attach to the trial neck components, wherein the heads are available in a variety of dimensional combinations that allow for incremental adjustments of the neck length; trialing the system in the patient; adjusting the rotational position of the hub with respect to the trial body without disengaging the trial neck from the hub; and retrialing the system in the patient.

In another form of the present invention, there is provided a method for trial implantation of a femoral hip prosthesis, comprising: providing a trial implant system comprising: a series of trial bodies incrementally sized for a range of patients; a hub that can be fixed to the trial body in a variety of rotational positions; a series of trial neck components that attach to the hub, wherein the necks are available in a variety of dimensional combinations that allow for incremental adjustment of horizontal offset, vertical offset or a combination of both horizontal offset and vertical offset; and a series of trial femoral heads that attach to the trial neck components, wherein the heads are available in a variety of dimensional combinations that allow for incremental adjustments of the neck length; trialing the system in the patient; adjusting the size of the trial neck without disrupting the orientation/rotational position of the hub with respect to the trial body; and retrialing the system in the patient.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIG. 1 is a schematic view illustrating a prior art trialing system;

FIG. 2 is a schematic view illustrating another prior art trialing system;

FIG. 3 is a schematic view illustrating another prior art trialing system;

FIG. 4 is a schematic view illustrating another prior art trialing system;

FIG. 5A is a schematic view illustrating another prior art trialing system;

FIG. 5B is a schematic sectional view taken along line 5B-5B of FIG. 5A;

FIG. 6A is a schematic view of a novel trial implant system formed in accordance with the present invention;

FIG. 6B is a schematic view of the hub element of the novel trial implant system shown in FIG. 6A;

FIG. 6B is a schematic view of the neck element of the novel trial implant system shown in FIG. 6A; and

FIG. 7 is a schematic view of another novel trial implant system formed in accordance with the present invention.

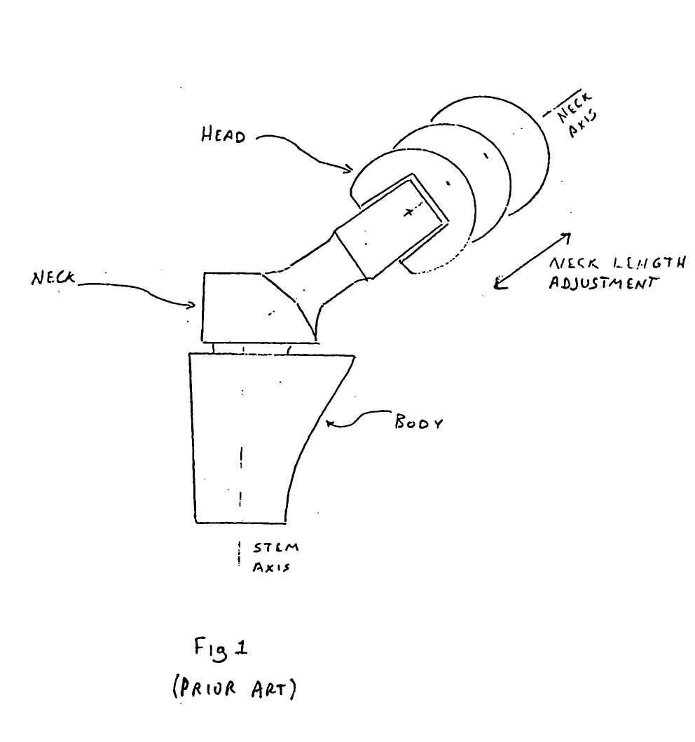

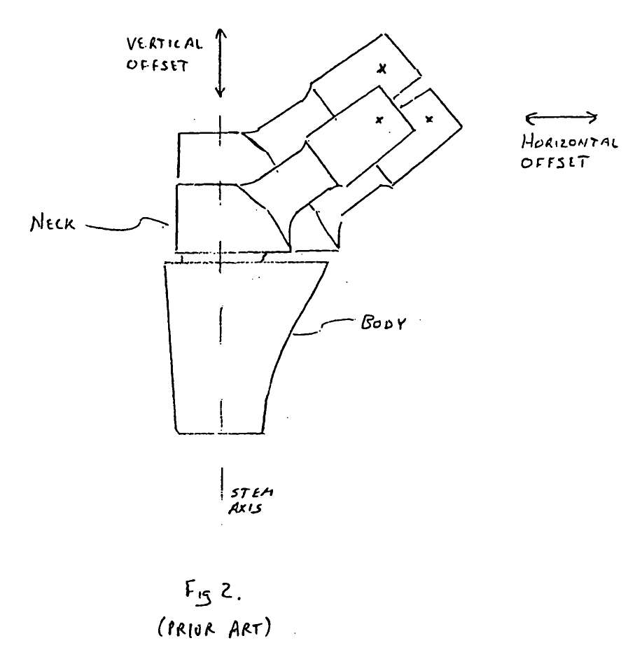

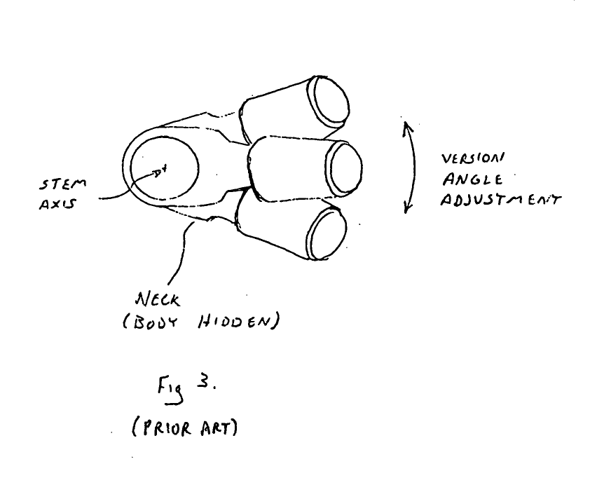

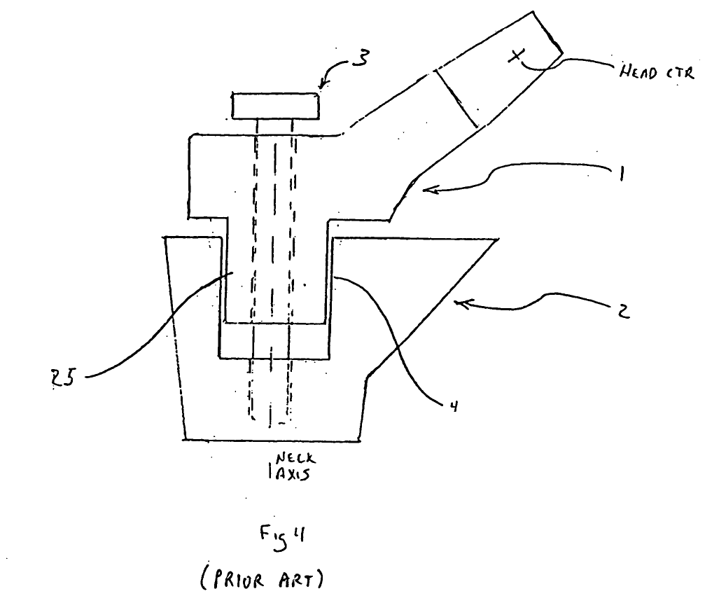

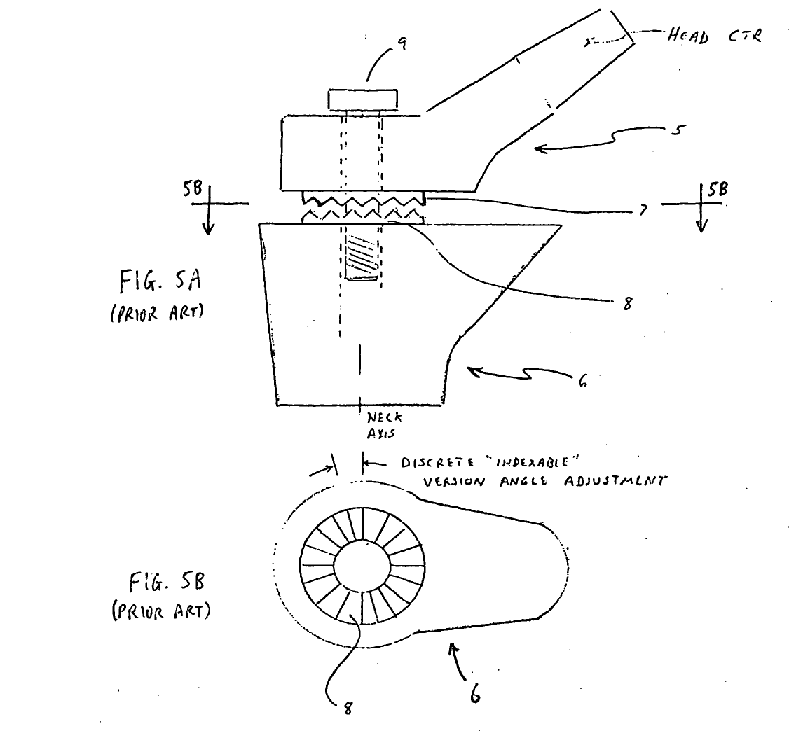

### DETAILED DESCRIPTION OF THE PREFERRED EMBODIMENTS

The present invention provides an improved trial implant system comprising:

(a) a series of trial bodies incrementally sized for a range of patients;

(b) a hub that can be fixed to the trial body in a variety of rotational positions;

(c) a series of trial neck components that attach to the hub, wherein the necks are available in a variety of dimensional combinations that allow for incremental adjustment of horizontal offset, vertical offset or a combination of both horizontal offset and vertical offset; and

(d) a series of trial femoral heads that attach to the trial neck components, wherein the heads are available in a variety of dimensional combinations that allow for incremental adjustments of the neck length.

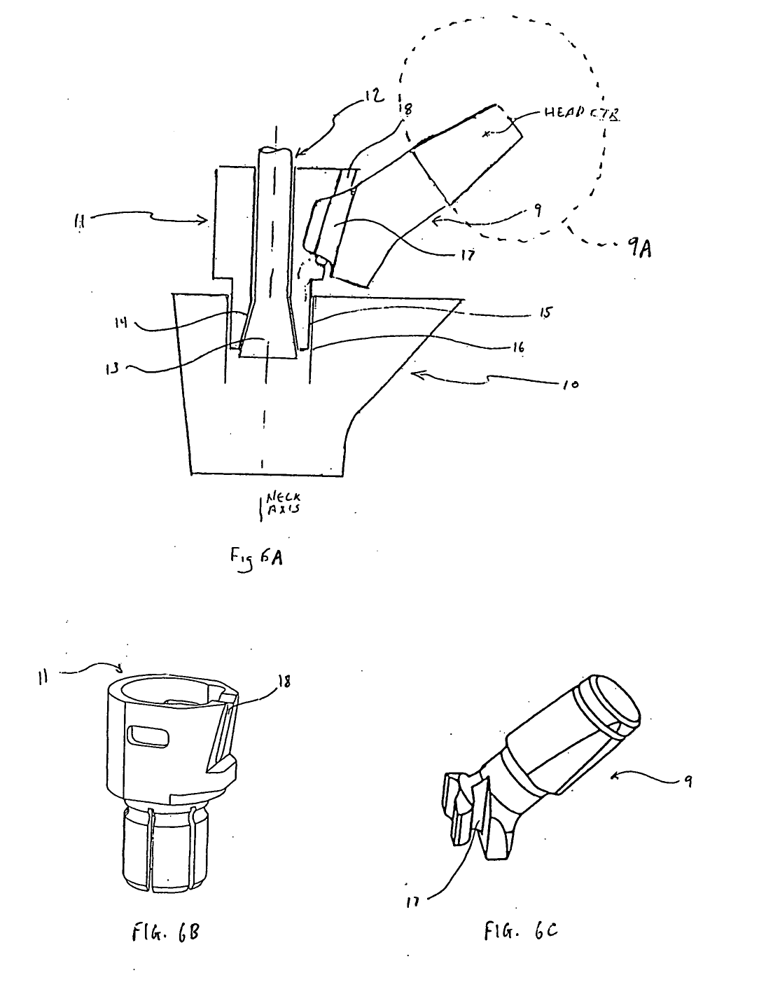

FIG. 6 shows one preferred embodiment of the present invention. The novel trial implant system is supplied with a series of necks 9 and trial bodies 10. The necks 9 attach to a hub 11 which in turn attaches to the trial body 10. Trial heads 9A attach to necks 9. In this embodiment, the hub 11 is inserted into the trial body 10. As the draw bolt 12 is drawn vertically, its flared end 13 engages a collet taper 14 on hub 11 which expands, causing the hub's outer surface 15 to engage the trial body's inner bore 16. The hub 11 and trial body 10 are then formed into a secure construct. To attach the neck 9, a tee boss 17 simply slides into a slot 18 on the hub 11. Trial heads 9A are press fit onto the outer end of neck 9.

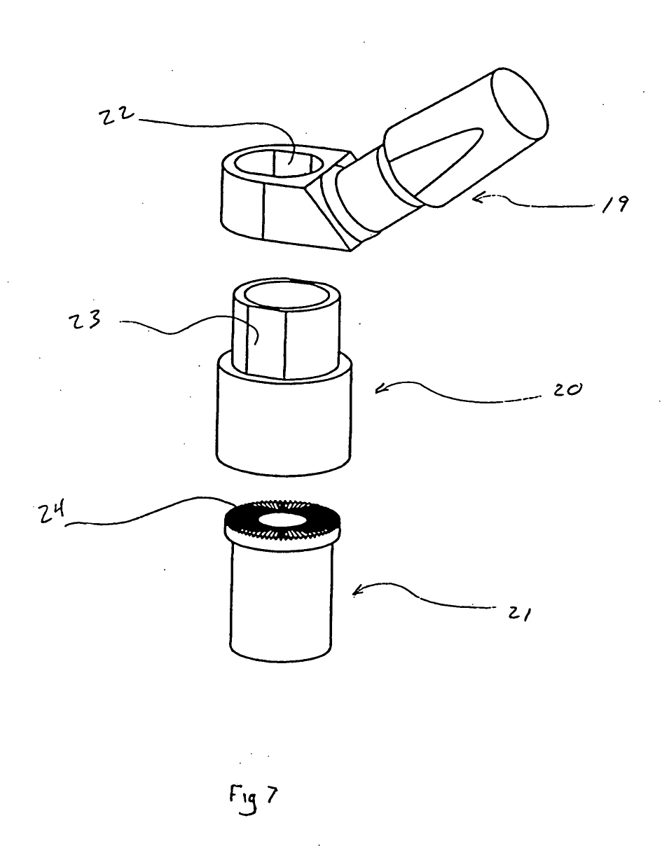

Another preferred embodiment of the present invention is shown in FIG. 7. In this embodiment, the trial neck 19 has opposing flats 22 that mate with counterpart flats 23 formed on the hub top 20. This construct then slides onto the hub bottom 21. The anteversion angle is locked by the interference of serrated surfaces 24 on the top of the hub bottom 21 and counterpart serrated surface (not shown) formed on the inside of the hub top 20. Hub 20 may be connected to trial body 10 using the draw bolt and collet taper arrangement described above with respect to the embodiment shown in FIGS. 6A-6C, and trial heads 9A may be fit onto the outer end of neck 19.

Still other embodiments of the present invention will be apparent to those skilled in the art in view of the present disclosure, and are considered to be withn the scope of the present invention.

What is claimed is:

1. A modular femoral hip implant trial device for providing independent adjustment of the anteversion angle, vertical offset, and horizontal offset thereof, the modular trial implant comprising:
a trial body component having a proximal end and a distal end, the trial body component defining a longitudinal axis between the proximal end and the distal end, the distal end of the trial body component being configured for placement within a bore in a bone, so that the cross-sectional edge of the trial body contacts the inner “endosteal” surface of the prepared femur, and a hub connection site configured at the proximal end of the trial body;
a hub component having a proximal end and a distal end, the hub defining a longitudinal axis between the proximal end and the distal end, a neck component connection site configured at the proximal end of the hub component, a trial body component connection site configured at the distal end of the hub component, and an anteversion angle adjustment mechanism for selectively adjusting the hub component from a first orientation to a second orientation, wherein the neck component connection site is adjusted from a first radial location to a second radial location with respect to its rotational position about the longitudinal axis of the trial body; and
a neck component having a proximal end and a distal end, a hub component connection site configured at the distal end of the neck component, and the proximal end of the neck component configured to receive a head thereon.

2. A modular femoral hip trial implant according to claim 1 wherein the hub component comprises a collet taper being extending into the distal end thereof, the collet taper being configured to receive a draw bolt with a flared end drawn therein so as to expand an outer surface of the hub component to engage an inner bore within the trial body component.

3. A modular femoral hip trial implant device according to claim 2 wherein the anteversion angle adjustment mechanism comprises loosening the draw bolt so as to permit rotation of the hub component about the longitudinal axis thereof and tightening the draw bolt so as to restrict rotation of the hub component about the longitudinal axis thereof.

4. A modular femoral hip trial implant device according to claim 1 wherein the hub component comprises a first portion and a second portion, the first portion having serrated surfaces comprising radially oriented serrations, said surfaces configured on a proximally facing section thereof, the second portion having serrated surfaces corresponding to the serrated surfaces of the first portion, the serrated surfaces of the second portion configured on a distally facing section of the second portion, and the serrated surfaces of the first portion and the serrated surfaces of the second portion being selectively engagable so as to (1) lock the first portion relative to the second portion and hence relative to the trial body component at a given anteversion angle when the serrated surfaces of the first portion and the serrated surfaces of the second portion are in engagement with one another, and (2) release the first portion relative to the second portion and hence relative to the trial body component for adjustment of the given anteversion angle to another anteversion angle when the serrated surfaces of the first portion and the serrated surfaces of the second portion are disengaged from one another.

5. A modular femoral hip trial implant device according to claim 1 wherein the trial body component connection site comprises a boss disposed in the hub component and the hub component connection site comprises a slot disposed in the trial body component and configured to mate with the boss.

6. A modular femoral hip trial implant device according to claim 1 wherein the trial neck component connection site comprises a slot disposed in the hub component and the hub component connection site comprises a tee boss disposed in the trial neck component and configured to mate with the slot.

7. A modular femoral hip trial implant device according to claim 1 wherein the trial body component connection site comprises a protrusion having a non-circular periphery disposed in the hub component and the hub component connection site comprises a recess formed in the trial body component and therein configured to mate with the protrusion of the trial body component connection site disposed in the hub component.

8. A modular femoral hip trial implant device according to claim 1 wherein the trial neck component connection site comprises a protrusion having a non-circular periphery disposed in the hub component and the hub component connection site comprises a recess formed in the trial neck component and therein configured to mate with the protrusion of the trial neck component connection site disposed in the hub component.

9. A modular femoral hip trial implant device according to claim 1 wherein a femoral head component is removably attached to the proximal end of the neck component.

10. A modular femoral hip trial implant device according to claim 1 wherein a femoral head component is fixedly attached to the proximal end of the neck component.

11. A modular femoral hip trial implant device according to claim 1 wherein the hub component and the trial body component are removably attached to one another.

12. The system of claim 1 wherein:
(a) the rotational position of a hub with respect to a trial body may be established or adjusted without disengaging the trial neck from the hub; and
(b) the appropriate size trial neck may be established or adjusted without disrupting the orientation/rotational position of a hub with respect to a trial body.

13. A modular femoral hip trial implant device according to claim 1 wherein the hub component and the trial body component are selectively movable relative to one another yet inseparable from one another.

14. A system for trial implantation of a modular femoral hip implant, the system providing independent adjustment of the anteversion angle, horizontal offset and vertical offset of the modular femoral implant, the system comprising:
a plurality of trial body components, each one of the trial body components having a proximal end and a distal end, the trial body component defining a longitudinal axis between the proximal end and the distal end, the distal end of the trial body component being configured for placement within a bore in a bone, and a hub connection site configured at the proximal end of the trial body;
a plurality of hub components, each one of the hub components having a proximal end and a distal end, the hub defining a longitudinal axis between the proximal end and the distal end, a neck component connection site configured at the proximal end of the hub component, a trial body component connection site configured at the distal end of the hub component, and an anteversion angle adjustment mechanism for selectively adjusting the hub component from a first orientation to a second orientation, wherein the neck component connection site is adjusted from a first radial location to a second radial location with respect to its rotational position about the longitudinal axis of the trial body; and
a plurality of neck components, each one of the neck components having a proximal end and a distal end, a hub component connection site configured at the distal end of the neck component, and the proximal end of the neck component configured to receive a head thereon;
wherein one from the plurality of trial body components is selected based on a geometry of a patient, one from the plurality of hub components is selected based on a geometry of a patient, and one from the plurality of neck components is chosen based on a geometry of a patient.

15. A system for trial implantation of a femoral hip prosthesis which allows for independent establishment and adjustment of anteversion, horizontal offset, and vertical offset, the system comprising a plurality of trial bodies, a hub, a plurality of trial necks and a plurality of trial femoral heads,
wherein each of the trial bodies register in a preformed cavity in the bone, and have a hub component connection site configured at the proximal end of the trial body component;
wherein the hub is configured to be rigidly fixed to one of the trial bodies in a plurality of rotational positions with respect to the trial body, and comprises a trial neck component connection site configured at the proximal end of the hub component,
wherein each of the trial necks comprises a hub component connection site configured at the distal end of the trial neck component, and has a geometry that reproduces one of the available horizontal and vertical neck offsets available for implantation, and includes a mechanism for receiving the trial femoral heads,
wherein each of the plurality of trial femoral heads has a mechanism for attaching to the trial necks, and has a geometry that reproduces the offsets of the femoral head implants.

16. The system of claim 15 wherein a hub is movable relative to, but inseparable from, a trial body.

17. The system of claim 15 wherein a hub is removable from a trial body.

18. The system of claim 15 wherein the hub can be fixed in discrete increments with respect to the trial body.

19. The system of claim 15 wherein the hub can have infinite orientation with respect to the trial body.

20. The system of claim 15 wherein a trial neck can be made integral with a trial femoral head.

21. The system of claim 15 wherein:
(a) the rotational position of a hub with respect to a trial body may be established or adjusted without disengaging the trial neck from the hub; and
(b) the appropriate size trial neck may be established or adjusted without disrupting the orientation/rotational position of a hub with respect to a trial body.

22. A method for trial implantation of a femoral hip prosthesis, comprising:
providing a trial implant system comprising:
a series of trial bodies incrementally sized for a range of patients;
a hub that can be fixed to the trial body in a variety of rotational positions;
a series of trial neck components that attach to the hub, wherein the necks are available in a variety of dimensional combinations that allow for incremental adjustment of horizontal offset, vertical offset or a combination of both horizontal offset and vertical offset; and
a series of trial femoral heads that attach to the trial neck components, wherein the heads are available in a variety of dimensional combinations that allow for incremental adjustments of the neck length;
trialing the system in the patient;
adjusting the rotational position of the hub with a respect to the trial body without disengaging the trial neck from the hub; and
retrialing the system in the patient.

23. A method for trial implantation of a femoral hip prosthesis, comprising:
providing trial implant system comprising:
a series of trial bodies incrementally sized for a range of patients;
a hub that can be fixed to the trial body in a variety of rotational positions;
a series of trial neck components that attach to the hub, wherein the necks are available in a variety of dimensional combinations that allow for incremental adjustment of horizontal offset, vertical offset or a combination of both horizontal offset and vertical offset; and
a series of trial femoral heads that attach to the trial neck components, wherein the heads are available in a variety of dimensional combinations that allow for incremental adjustments of the neck length;
trialing the system in the patient;
adjusting the horizontal offset, vertical offset, or combined offset of the trial neck without disrupting the orientation/rotational position of the hub with respect to the trial body; and
retrialing the system in the patient.
